(19) United States
(12) Patent Application Publication
(10) Pub. No.: US 2009/0099531 A1
(43) Pub. Date: Apr. 16, 2009

(54) Packaging for selectivity lubricating part of a medical device

(76) Inventor: Henry Louis Griesbach, III, Atlanta, GA (US)

Correspondence Address: KIMBERLY-CLARK WORLDWIDE, INC.;Catherine E. Wolf, 401 NORTH LAKE STREET, NEENAH, WI, 54956, US

(21) Appl. No.: 11/974,612

(22) Filed: Oct. 15, 2007

(51) Int. Cl. A61M 25/01; B65D 17/00

(52) U.S. Cl. 604/265; 206/571

(57) ABSTRACT

A medical device lubricating assembly for imparting a lubricious and/or antimicrobial liquid onto a medical device is provided. The medical device is surrounded by a pouch and the assembly includes a barrier for bifurcating the pouch into a wet compartment and a dry compartment. Lubricating fluid is stored in the wet compartment or is otherwise introduced into the wet compartment prior to removal of the medical device from the pouch. This assists in insertion and implantation of the device into a body cavity or opening.

### BACKGROUND

Medical devices adapted to be inserted into body cavities are commonly used. These devices are often enclosed in permeable or impermeable packages for shipment, storage, and protection from contamination from the outside environment. However, many of these devices may be difficult to insert into body cavities upon removal from the packages.

Many prior art devices have attempted to overcome this problem by prelubricating the device before packaging. However, many of these prelubricated devices do not contain barriers for separating the medical device into wet in dry components. In this regard, upon removal of a medical device from a package, a clinician may undesirably rub lubricant onto his hands decreasing the effectiveness of his grip on the medical device during implantation and removal. Further, many of the prior art devices require that the medical device be precoated with an adherent swellable material and then coated again with an activating lubricant once it is ready to be removed from the package. This results in an unnecessary added expense.

Additionally, many of these prelubricated or unlubricated devices, may be become contaminated when they are removed from the package and inserted into a body cavity or opening. Although the device is generally provided to the health professional sterile, it is removed and handled in a non-sterile environment. In this regard, a health professional may contaminate the device with his hand or body, microbes from the outside environment may contaminate the medical device, or the medical device may accidentally come into contact with a contaminated surface prior to insertion into a body cavity or opening.

Contamination is of particular concern because when devices such as endotracheal tubes are inserted into the trachea, the likelihood of Ventilator Associated Pneumonia (VAP) increases.

Thus, there remains a need for an economical medical device lubricating assembly which provides for lubrication of the medical device for prevention of tissue irritation and bacterial biofilm formation. Additionally, there is a need for a medical device lubricating assembly which separates the lubricated wet compartment containing the end of the medical device adapted for insertion into a body cavity or opening from the dry compartment which encloses the end of the medical device which the clinician handles when inserting and removing the device from a body cavity or opening. There is also a need for an economical medical device assembly which reduces the possibility of contamination of the medical device immediately prior to insertion into a body cavity or opening.

### SUMMARY OF INVENTION

The present invention provides for a medical device lubricating assembly. The medical device lubricating assembly includes a medical device adapted to be coated with a liquid and having a distal and proximal portion. The assembly also includes a pouch which includes a thin flexible region and surrounds the medical device. The pouch may be plastic and/or impermeable to liquids and is bifurcated into a wet compartment and a dry compartment. Additionally, the medical device assembly includes a barrier located inside the pouch. The barrier may be a film and it surrounds at least a portion of the medical device and is in communication with both the pouch and medical device. Further, a lubricating fluid may be within the wet compartment of the pouch and in communication with the distal portion of the medical device.

Desirably, the medical device may be an airway tube such as an endotracheal tube or tracheotomy tube, an internal feeding tube, a urinary catheter, a blood catheter, an aspirating catheter, or another type of intracorporeal dwelling catheter. The lubricating fluid in the wet compartment of the pouch may be a polymeric solution such as a phospholipid, collagen, laminin, polyamino acids, carboxymethylcellulose, polyvinylpyrrolidone, chitosan, polyvinylpyroolidone-co-vinyacetate, polyethylene glycol, or pluronics. Additionally, it may be water, oil, an oil-in-water emulsion, a salt solution, or a colloidal suspension. Further, the lubricating fluid may include an active agent such as triclosan, chlorohexidine, charged silver, polyhexamethylene biguanide or may include a surfactant or viscosity modifier. The medical device may be uncoated prior to contact with lubricating fluid from the wet compartment.

Another aspect of the invention also provides for a lubricating medical device assembly. The medical device lubricating assembly includes a medical device adapted to be coated with a liquid and having a distal and proximal portion. The assembly also includes a pouch which includes a thin flexible region and surrounds the medical device. The pouch may be plastic and/or impermeable and is bifurcated into a wet compartment and a dry compartment. Additionally, the medical device assembly includes a barrier located inside the pouch. The barrier may be a film, sponge, foam, nonwoven, brush, or polymer ring and it surrounds at least a portion of the medical device and is in communication with both the pouch and medical device. Further, a means for lubricating the wet compartment of the pouch with a lubricating fluid is provided.

Desirably, the means for wetting the wet compartment of the pouch is a frangible container or an access port. When a frangible container is used, the frangible container encapsulates the lubricating fluid and is adapted to coat the portion of the medical device in the wet compartment of the pouch upon rupture of the frangible container. Rupturing may be accomplished by applying pressure to the frangible container by hands, finger, or other effective means. Additionally, when an access port is used as a means for wetting the wet compartment, the access port may be an aperture, slit, perforation, or one way valve wherein lubricating fluid may be introduced into the wet compartment of the pouch to lubricate the distal portion of the medical device prior to insertion into a body cavity or opening.

Yet another aspect of the invention provides for a medical device lubricating assembly. The medical device lubricating assembly includes a medical device adapted to be coated with a liquid and having a distal and proximal portion. The assembly also includes a pouch which includes a thin flexible region and surrounds the medical device. The pouch is bifurcated into a wet compartment and a dry compartment. Additionally, the medical device assembly includes an absorbent barrier located inside the pouch. The absorbent barrier may be a sponge, foam, nonwoven, or fibers and it surrounds at least a portion of the medical device and is in communication with both the pouch and medical device. Further, a means for lubricating at least a portion of the absorbent barrier is provided.

Desirably, the means for wetting at least a portion of the absorbent barrier is a frangible container. The frangible container may be located within the absorbent barrier, may encapsulate liquid, and may introduce liquid into the absorbent barrier upon rupture of the frangible container.

Another aspect of the invention provides for a method for lubricating at least a portion of a medical device immediately prior to insertion into a body cavity or opening. The method includes providing a medical device lubricating assembly which includes a medical device adapted to be coated with a lubricating fluid and having a distal portion and a proximal portion; a pouch enclosing the medical device and including a thin flexible material; an absorbent barrier bifurcating the pouch into a wet and dry compartment and surrounding and being in communication with the medical device; and a frangible container located within the absorbent barrier and having encapsulated lubricating fluid.

The method further includes rupturing the frangible container so that lubricating fluid is introduced into the absorbent barrier; removing the medical device so that the distal portion of the medical device is wiped with lubricating fluid upon removal of the medical device from the pouch; and inserting the medical device into a body cavity or opening.

Yet another aspect of the invention provides for a method of providing a system for lubricating at least a portion of a medical device immediately prior to insertion into a body cavity or opening. The system includes providing a medical device lubricating assembly which includes a medical device adapted to be coated with a lubricating fluid and having a distal portion and a proximal portion; a pouch enclosing the medical device and including a thin flexible material; an absorbent barrier bifurcating the pouch into wet and dry compartment and surrounding and being in communication with the medical device; and a frangible container located within the absorbent barrier and having encapsulated lubricating fluid.

The system further includes providing a means for rupturing the frangible container so that lubricating fluid is introduced into the absorbent barrier; providing a means for removing the medical device so that the distal portion of the medical device is wiped with lubricating fluid upon removal of the medical device from the pouch; and providing a means for inserting the medical device into a body cavity or opening.

### BRIEF DESCRIPTION OF DRAWINGS

FIG. 1 is a perspective view of a medical device lubricating assembly having a wet compartment and dry compartment.

FIGS. 2A and 2B are perspective views of rupturing mechanisms for the barrier of a medical device lubricating assembly.

FIGS. 3A and 3B are perspective view of mechanisms for separating barrier components upon removal of a medical device from a medical device lubricating assembly.

FIG. 4 is a perspective view of a medical device lubricating assembly having a frangible container within its wet compartment.

FIG. 5 is a perspective view of a medical device lubricating assembly having an access port for introduction of fluid into the wet compartment of a medical device lubricating assembly.

FIGS. 6A and 6B are perspective views of a wiping mechanism utilizing an absorbent barrier.

FIG. 7 is a perspective view of a wiping mechanism utilizing an absorbent barrier having a frangible reservoir embedded within it.

FIG. 8 is a cross-sectional view of an absorbent barrier having a frangible reservoir embedded within it.

### DETAILED DESCRIPTION

The medical device lubricating assemblies of the present invention provide for medical device lubricating assemblies incorporating medical devices having various means for delivering liquids to the wet compartment of a pouch containing a medical device and the distal portion of a medical device. These liquids provide lubricious properties and/or antimicrobial protection for the medical devices.

The invention will be described with reference to the following description and figures which illustrate certain embodiments. It will be apparent to those skilled in the art that these embodiments do not represent the full scope of the invention which is broadly applicable in the form of variations and equivalents as may be embraced by the claims appended hereto. Furthermore, features described or illustrated as part of one embodiment may be used with another embodiment to yield still a further embodiment. It is intended that the scope of the claims extend to all such variations and embodiments.

In the interests of brevity and conciseness, any ranges of values set forth in this specification contemplate all values within the range and are to be construed as support for claims reciting any sub-ranges having endpoints which are whole number values within the specified range in question. By way of a hypothetical illustrative example, a disclosure in this specification of a range of from 1 to 5 shall be considered to support claims to any of the following ranges: 1-5; 1-4; 1-3; 1-2; 2-5; 2-4; 2-3; 3-5; 3-4; and 4-5.

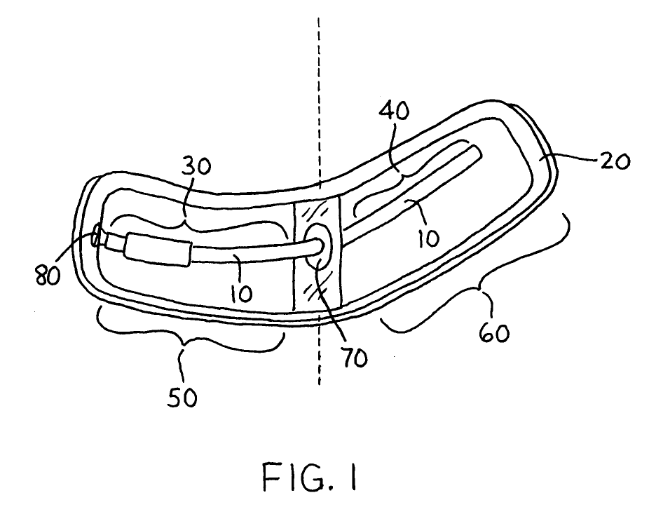

Referring to FIG. 1, a medical device lubricating assembly is provided. The medical device lubricating assembly includes a medical device 10 having distal 30 and proximal 40 portions. The distal portion of the medical device corresponds to a wet compartment 50 within the pouch 20 and the proximal portion 60 of the medical device corresponds to a dry compartment within the pouch 20. The medical device may be any medical device that may is adapted to be inserted into a body cavity or opening. These devices include, but are not limited to, endotracheal tube, tracheotomy tube, internal feeding tube, urinary catheters, blood catheters, aspirating catheters or any other intracorporeal dwelling catheter. It is also contemplated that medical devices for use with the present invention may also include devices which are not designed for insertion into a body cavity or opening.

Returning to FIG. 1, a pouch 20 surrounds the medical device. The pouch may include any material capable of providing some degree of protection against the outside atmosphere during shipment or storage of the medical device. These materials include, but are not limited to, plastic. The pouch 20 may be permeable or impermeable to gases, though desirably the pouch may be gas permeable so it may be sterilized. The pouch may be any size or shape which enables the medical device to be fully enclosed, for example, square or rectangular. Additionally, a second pouch or covering is not needed to surround the medical device in order to maintain the position of the medical device within the pouch. In this regard, an optional plug or connector 80 may be integrated with the packaging which is adapted to connect with the distal end of the medical device and hold it in place within the pouch.

The medical device assembly includes a barrier 70 surrounding at least a portion of the medical device. Desirably, the barrier will act as a divider between the wet 50 and dry 60 compartments of the pouch and will be in communication with the pouch and the medical device in a zone of contact with the device. The barrier may be constructed of the same material as the pouch, i.e. plastic, or may be a sponge, nonwoven, film, foam or brush and the barrier may be configured as a ring that encircles the medical device. However, any material suitable or configuration effective for containing liquid during within the wet compartment storage and shipping of the medical device assembly may also be used.

Returning to FIG. 1, various liquids may be present within the wet compartment of the pouch (which corresponds to the distal portion of the medical device). These liquids may act as a lubricious coating for the distal portion of the device to aid in insertion of the medical device into a body cavity or opening. The presence of the lubricious coating decreases the chances that insertion will result in irritation of the tissues of the body cavity or opening and an increase in the likelihood of entrance of pathogens into the body through the delicate tissues of the body cavity or opening. In this regard, the coatings are designed to provide short term protection, i.e. the time required implantation of the device. This short term protection may range from about 1 minute up to about 2 weeks, desirably between 1 minute and 1 hour. It is also contemplated that the short term protection may last longer than 2 weeks. Desirably, after implantation, the coating or coatings will be flushed away by the natural secretions of the body.

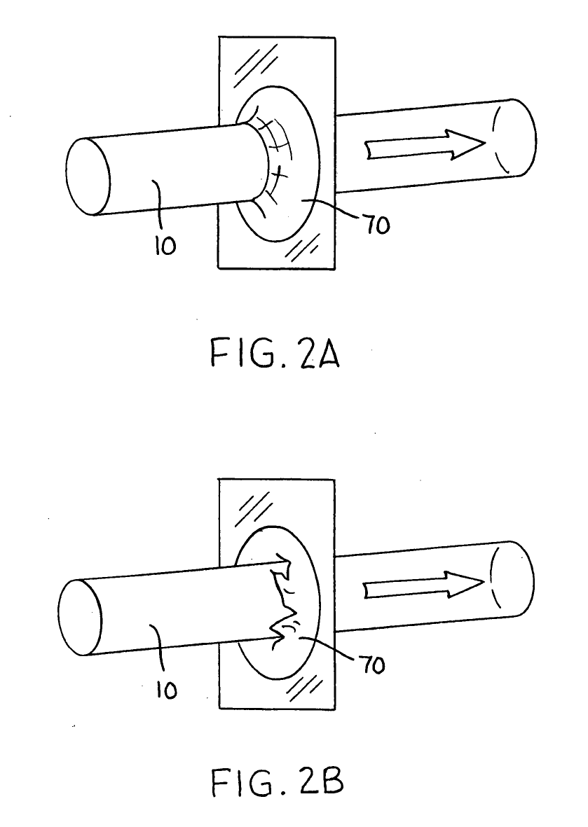

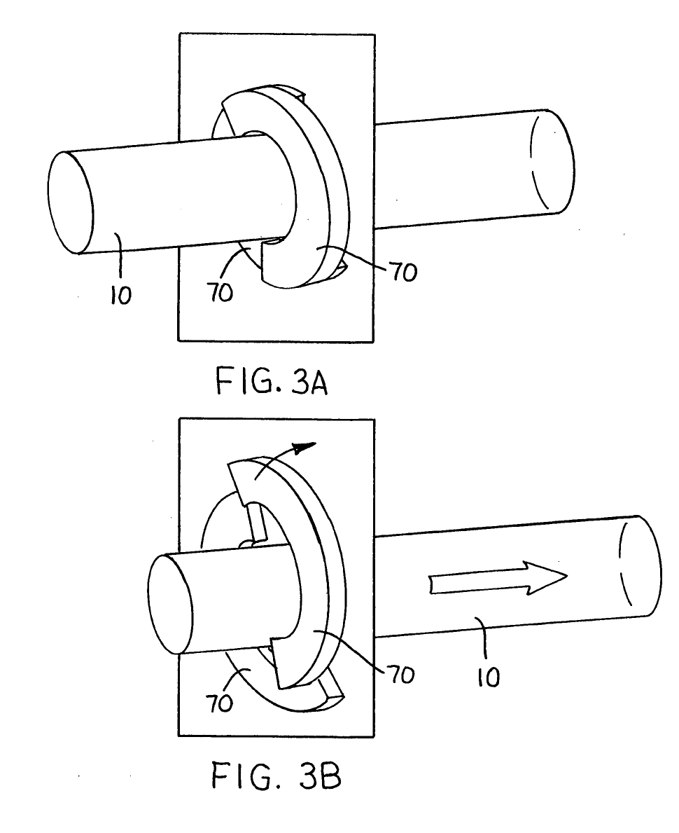

The barrier dividing the wet compartment and dry compartment desirably defines an aperture which surrounds the medical device during packaging, shipping, and storing. Upon removal of the medical device from the package immediately prior to use, the barrier may be ruptured to allow the medical device to be removed from the pouch (See. FIGS. 2A and 2B). Alternatively, the barrier may contain separable components which detach when the proximal portion of the medical device is removed from the pouch (See FIG. 3A). Thus, it is desirable that the barrier be strong enough to prevent liquid from traveling from the wet compartment to the dry compartment during storage, but flexible enough that the medical device can be removed from the barrier immediately prior to insertion of the medical device into a body cavity or opening.

In addition to use as a lubricious coating, the coatings may be used to impart antibacterial and anti-microbial properties to the medical device. In this regard, certain liquids may prevent bacteria from forming on the surface of the medical device prior to insertion into the body cavity or opening and/or kill microbes and bacteria that colonize on the medical device prior to insertion in the body cavity or opening. Further, even after insertion into the body cavity or opening, some liquids may deactivate bacteria and microbes and decrease incidences of VAP. Desirably, when the lubricious coating has antimicrobial or antibacterial properties, it will also offer short term protection against microbes from the environment. As previously mentioned, short term protection may range from about 1 minute up to about 2 weeks, desirably between 1 minute and 1 hour. It is also contemplated that the short term protection may last longer than 2 weeks.

Importantly, whether a liquid has anti-microbial properties or not, each of these liquids does not require the medical device to have an adherent swellable coating on its body prior to contact with a lubricious coating, unlike some prior art medical devices. This decreases the economic costs of providing lubrication and/or antibacterial protection for the medical device. Additionally, because in some situations the pouch may have some degree of permeability, microbes may enter the pouch through the atmosphere during shipment or storage. If a coating is adhered to the surface of the medical device during this time, microbes may attach to the adhered surface and colonize within the coating. By utilizing a medical device which is stored in a lubricious coating but does not have an adherent swellable coating on its surface, the possibility of bacterial biofilm formation during storage and shipment is significantly reduced.

Lubricants suitable for use with the present invention include polymeric solutions. Non-limiting examples of polymeric solutions include phospholipid, acrylic acid polymer, collagen, laminin, polyamino acids, carboxymethylcellulose, polyvinylpyrrolidone, chitosan, polyvinylpyroolidone-co-vinyacetate, polyethylene glycol, pluronics, or combinations thereof.

Additionally, various other lubricants may be used including silicon based water soluble lubricants such as alkylene oxide modified silicone glycol. Further, lubricants such as lecithin, water, water soluble gums, saline solutions, oil, colloidal suspensions, and emulsions including oil-water-emulsions may be used. Any lubricant, however, which is biocompatible and which provides a lubricious coating to the medical device for insertion into a body cavity or opening is appropriate.

Regardless, of the type of lubricant used, various additives may be used with the lubricating solutions. For example, active agents, with optional surfactants, may be added to the lubricating solution. The active agents kill or deactivate microbes and bacteria upon contact or within a short period of time. These active agents include, but are not limited to, triclosan, chlorohexidine, charged silver, polyhexamethylene biguanide, and combinations thereof. Surfactants may be used with active agents to aid in the profusion of the active agent into the microbial or bacterial cell wall, thus aiding the active agent's ability to kill or deactivate the microbe or bacteria. Non-limiting examples of surfactants include copolymers of polysiloxane and polyoxyethylene.

Viscosity modifiers, as known in the art, may also be used with lubricants. Viscosity modifiers may function to reduce surface friction of and form a water multilayer that may reduce the affinity for bacteria to attach to the medical device.

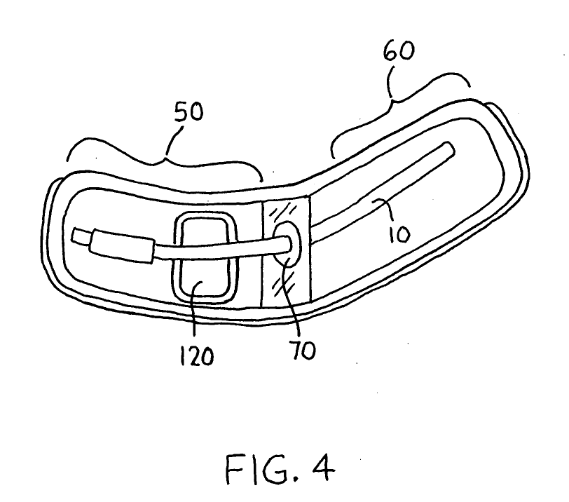

Turning to FIG. 4, desirably, a frangible container 120, adapted to lubricate the distal portion 50 of the medical device 10, may be located within the wet compartment 30 of the pouch. The frangible container may be constructed of the same material of pouch, i.e. plastic, or may be constructed of any material suitable during storage and shipment of the medical device lubricating assembly.

It is contemplated that upon rupture of the frangible container, liquid may be delivered directly to the portion of the medical device in the wet compartment, i.e, the distal portion. Upon contact with the distal portion of the medical device the liquid will act as a lubricant and/or a lubricant having anti-microbial properties as discussed above. To remove the partially lubricated medical device from the pouch, the care giver opens the proximal end of the pouch, grasps the proximal end of the device and removes it from the pouch to insert the coated distal portion into the body cavity or opening. Although it is desirable to coat the distal portion of the medical device because it is generally inserted into the body cavity or opening, it is also contemplated that any portion of the medical device may be coated.

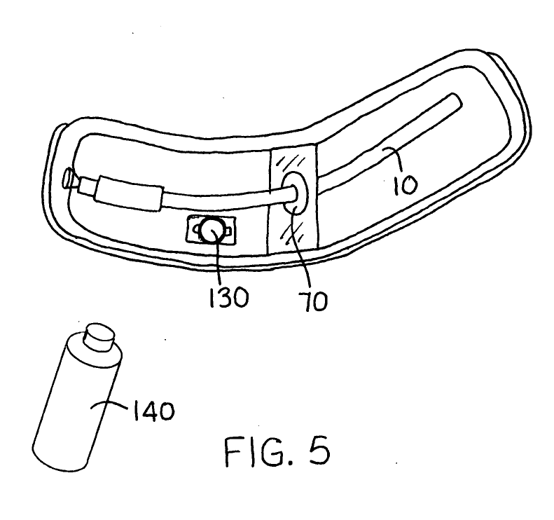

Alternatively, referring to FIG. 5, the lubricant 140 may be introduced into the wet compartment by way of an access port 130. In this regard, lubricant from a container or through other means may be introduced into the access port. Upon introduction of the lubricant into the access port, the lubricant will coat the distal portion of the medical device in the wet compartment. The medical device may then be removed from the pouch by a care giver and inserted into the body cavity or opening.

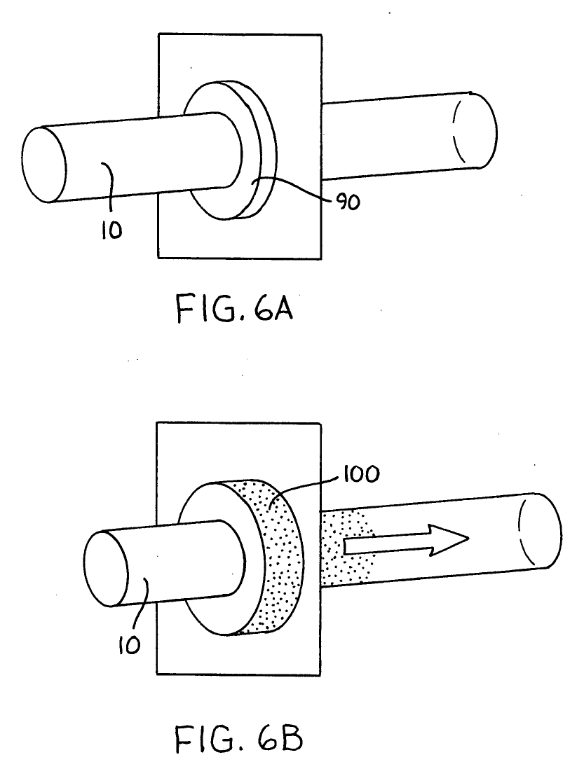

Turning to FIGS. 6A and 6B, it may also be desirable that the bifurcating barrier be an absorbent barrier 90. Upon introduction of fluid into the wet compartment, the fluid may flow into the absorbent barrier 100. The absorbent barrier may then function to lubricate the distal portion of the medical device as it is removed from the pouch or alternatively, it may wipe excess fluid from the medical device while maintaining a lubricious coat. In situations where the absorbent barrier wipes excess lubricant from the medical device, the absorbent barrier advantageously prevents excess fluid from falling on the floor, which could create potentially hazardous conditions within the medical procedure room.

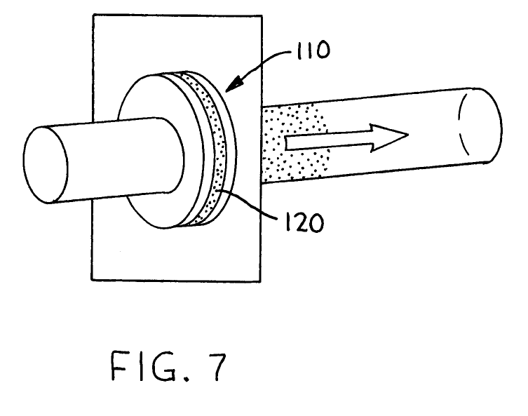

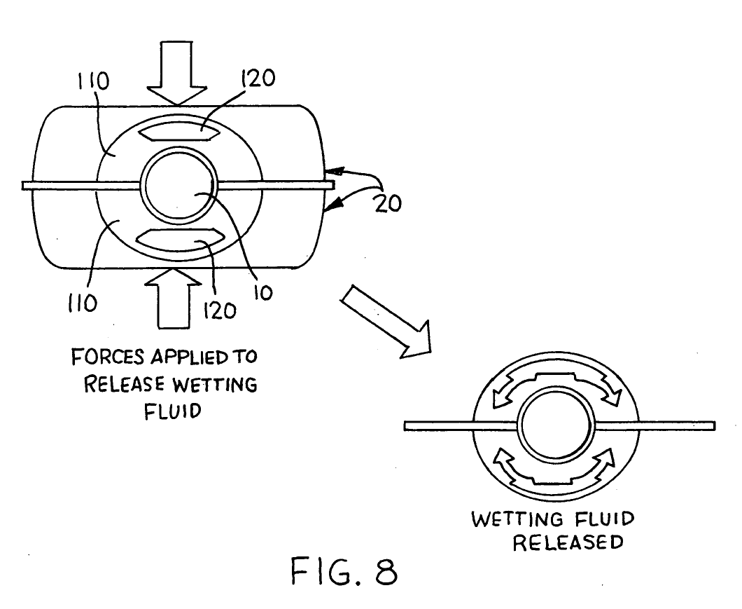

Referring to FIGS. 7 and 8, desirably, an absorbent barrier may not only provide a wiping function but may also serve as a reservoir for a lubricating fluid. In this regard, the absorbent barrier may have a frangible reservoir inside it 110, 120. Upon application of force to the absorbent barrier the frangible reservoir ruptures and fluid is released into the absorbent barrier. The absorbent barrier may then perform the wiping function as described above. Advantageously, the combination reservoir function and wiper function limits or eliminates free flowing liquid during storage of the pouch. This is particularly important in situation where potentially volatile liquids are found within the pouch or situations where a stand alone frangible container may potentially rupture during storage or transport.

In addition to the medical device lubricating assemblies and methods provided above, the present invention encompasses a method of providing a system for coating at least a portion of a medical device immediately prior to insertion into a body cavity or opening.

Generally speaking, the system includes providing a medical device lubricating assembly which includes a medical device adapted to be coated with a lubricating fluid and having a distal portion and a proximal portion; providing a pouch enclosing the medical device and including a thin flexible material; providing an absorbent barrier bifurcating the pouch into wet and dry compartment and surrounding and being in communication with the medical device; and providing a frangible container located within the absorbent barrier and having encapsulated lubricating fluid.

The system further includes providing a means for rupturing the frangible container so that lubricating fluid is introduced into the absorbent barrier; providing a means for removing the medical device so that the distal portion of the medical device is wiped with lubricating fluid upon removal of the medical device from the pouch; and providing a means for inserting the medical device into a body cavity or opening.

What is claimed is:

1. A medical device lubricating assembly comprising:
A medical device adapted to be coated with a lubricating fluid and having a distal and proximal portion;
a pouch comprising a thin flexible material, the pouch bifurcated into a wet compartment and dry compartment;
a barrier in communication with and surrounding at least a part of the medical device, the barrier being adapted to separate the wet compartment from the dry compartment; and
a lubricating fluid within the wet compartment and in communication with the distal portion of the medical device.

2. The medical device lubricating assembly of claim 1, wherein the medical device is an airway tube, internal feeding tube, urinary catheter, blood catheter, aspirating catheter, or other intracorporeal dwelling catheter.

3. The medical device lubricating assembly of claim 1, wherein the pouch is gas impermeable.

4. The medical device lubricating assembly of claim 1, wherein the pouch comprises plastic.

5. The medical device lubricating assembly of claim 1, wherein the medical device is uncoated prior to contact with the lubricating fluid within the wet compartment.

6. The medical device lubricating assembly of claim 1, wherein the lubricating fluid comprises water, oil, or oil-in-water emulsion.

7. The medical device lubricating assembly of claim 6, wherein the lubricating fluid further comprises a viscosity modifier.

8. The medical device lubricating assembly of claim 1, wherein the barrier comprises a film.

9. A medical device lubricating assembly comprising:
A medical device adapted to be coated with a lubricating fluid and having a distal and proximal portion;
a pouch comprising a thin flexible material, the pouch bifurcated into a wet compartment and dry compartment;
a liquid impermeable barrier in communication with and surrounding the medical device, the barrier being adapted to separate the wet compartment from the dry compartment; and
a means for lubricating the wet compartment of the pouch with a lubricating fluid.

10. The medical device lubricating assembly of claim 9, wherein the medical device is an airway tube, internal feeding tube, or catheter.

11. The medical device lubricating assembly of claim 9, wherein the pouch is gas impermeable.

12. The medical device lubricating assembly of claim 9, wherein the pouch comprises plastic.

13. The medical device lubricating assembly of claim 9, wherein at least a portion of the medical device is uncoated prior to contact with the lubricating fluid.

14. The medical device lubricating assembly of claim 9, wherein the lubricating fluid comprises a viscosity modifier.

15. The medical device lubricating assembly of claim 9, wherein the barrier comprises a film, a sponge, a foam, a nonwoven, or polymer ring that is attached to the pouch.

16. The medical device lubricating assembly of claim 9 wherein the means for lubricating the wet compartment is a frangible container.

17. The medical device lubricating assembly of claim 16 wherein the frangible container encapsulates the lubricating fluid and is adapted to coat the portion of the medical device in the wet compartment upon rupture of the frangible container.

18. The medical device lubricating assembly of claim 9 wherein the means for lubricating the wet compartment is an access port.

19. The medical device lubricating assembly of claim 18, wherein the access port is an aperture, slit, perforation, or one way valve.

20. The medical device lubricating assembly of claim 19 wherein the access port is adapted to introduce a lubricating fluid located outside the pouch into the wet compartment of the pouch.

21. The medical device lubricating assembly of claim 9 wherein the lubricating fluid comprises a polymeric solution.

22. The medical device assembly of claim 21, wherein the polymeric solution is a phospholipid, collagen, laminin, polyamino acids, carboxymethylcellulose, polyvinylpyrrolidone, chitosan, polyvinylpyroolidone-co-vinyacetate, polyethylene glycol, pluronics, or combinations thereof.

23. The medical device lubricating assembly of claim 9 wherein the lubricating fluid comprises an aqueous salt solution, emulsion, or colloidal suspension.

24. The medical device assembly of claim 9, wherein the lubricating fluid comprises an active agent.

25. The medical device assembly of claim 24, wherein the active agent is trichlosan, chlorohexidine, charged silver, polyhexamethylene biguanide, or combinations thereof.

26. The medical device assembly of claim 9, wherein the lubricating fluid comprises a surfactant.

27. The medical device assembly of claim 9, wherein the means for lubricating the wet compartment is located within the wet compartment.

28. A medical device lubricating assembly comprising:
A medical device adapted to be coated with a lubricating fluid and having a distal and proximal portion;
a pouch comprising a thin flexible material, the pouch bifurcated into a wet compartment and dry compartment;
an absorbent barrier in communication with and surrounding the medical device, the barrier being adapted to separate the wet compartment from the dry compartment; and
a means for lubricating at least a portion of the absorbent barrier with a lubricating fluid.

29. The medical device lubricating assembly of claim 28, wherein the medical device is an airway tube, internal feeding tube, or catheter.

30. The medical device lubricating assembly of claim 28, wherein the pouch is gas impermeable.

31. The medical device lubricating assembly of claim 28, wherein the pouch comprises plastic.

32. The medical device lubricating assembly of claim 28, wherein the medical device is uncoated prior to contact with the lubricating fluid.

33. The medical device lubricating assembly of claim 28, wherein the lubricating fluid comprises a viscosity modifier.

34. The medical device lubricating assembly of claim 28, wherein the absorbent barrier comprises a foam, sponge, nonwoven, or cellulose fibers.

35. The medical device lubricating assembly of claim 28, wherein the means for lubricating the absorbent barrier is a frangible container.

36. The medical device lubricating assembly of claim 35, wherein the frangible container is located within the absorbent barrier.

37. The medical device lubricating assembly of claim 36, wherein the frangible container encapsulates the lubricating fluid and is adapted to introduce lubricating fluid into the absorbent barrier upon rupture of the frangible container.

38. The medical device lubricating assembly of claim 28 wherein the lubricating fluid comprises a polymeric solution.

39. The medical device assembly of claim 38, wherein the polymeric solution is a phospholipid, collagen, laminin, polyamino acids, carboxymethylcellulose, polyvinylpyrrolidone, chitosan, polyvinylpyroolidone-co-vinyacetate, polyethylene glycol, pluronics, or combinations thereof.

40. The medical device assembly of claim 26, wherein the lubricating fluid comprises a surfactant.

41. A method for lubricating at least a portion of a medical device immediately prior to insertion into a body cavity or opening, the method comprising:
Providing a medical device lubricating assembly comprising:
A medical device adapted to be coated with a lubricating fluid and having a distal and proximal portion;
a pouch enclosing the medical device and comprising a thin flexible material, the pouch bifurcated into a wet compartment and dry compartment;
an absorbent barrier in communication with and surrounding the medical device, the barrier being adapted to separate the wet compartment from the dry compartment; and
a frangible container having encapsulated lubricating fluid and located within the absorbent barrier;
Rupturing the frangible container so that the lubricating fluid is introduced into the absorbent barrier;
Removing the medical device so that the distal portion of the medical device is wiped with lubricating fluid upon removal of the medical device from the pouch; and
Inserting the medical device into a body cavity or opening.

42. A method of providing a system for lubricating at least a portion of a medical device immediately prior to insertion into a body cavity or opening, the system comprising:
Providing a medical device lubricating assembly comprising:
A medical device adapted to be coated with a lubricating fluid and having a distal and proximal portion;
a pouch enclosing the medical device and comprising a thin flexible material, the pouch bifurcated into a wet compartment and dry compartment;
an absorbent barrier in communication with and surrounding the medical device, the barrier being adapted to separate the wet compartment from the dry compartment; and
a frangible container having encapsulated lubricating fluid therein, the frangible container located within the absorbent barrier;
Providing a means for rupturing the frangible container so that the lubricating fluid is introduced into the absorbent barrier;
Providing a means for removing the medical device so that the distal portion of the medical device is wiped with lubricating fluid upon removal of the medical device from the pouch; and
Providing a means for inserting the medical device into a body cavity or opening.
